(19) United States
(12) Patent Application Publication
(10) Pub. No.: US 2013/0153461 A1
(43) Pub. Date: Jun. 20, 2013

(54) HAIR TREATMENT ACCESSORY

(71) Applicants: Duane Charles Hein, Monroe, CT (US); Vito James Carlucci, Stratford, CT (US)

(72) Inventors: Duane Charles Hein, Monroe, CT (US); Vito James Carlucci, Stratford, CT (US)

(21) Appl. No.: 13/326,926

(22) Filed: Dec. 15, 2011

(51) Int. Cl. A45D 6/00; A61Q 5/12; A61K 8/02

(52) U.S. Cl. 206/581; 424/401; 424/70.1; 132/221

(57) ABSTRACT

A hair treatment accessory for use with a hair styling apparatus includes a substrate base for mounting to a hair styling apparatus and a treatment agent disposed on the substrate base. The treatment agent is applied to one of the hair or skin of a subject during use of the hair styling apparatus. The treatment agent may include a conditioning, strengthening, repairing or revitalizing fluid. The treatment agent may include a conditioning oil. The conditioning oil may include ARGAN oil with or without silicon. The substrate base may include an irregular surface dimensioned to assist in retention of the treatment agent on the substrate base. The irregular surface may include open cells or channels. The irregular surface may be formed through a sintering process.

### BACKGROUND

1. Technical Field

The present disclosure relates to an apparatus for treating hair, and, more particularly, relates to an accessory for a hair styling apparatus in which the accessory is infused with a medicinal/conditioner agent for dispensing on the hair to strengthen, repair, revitalize and/or condition hair during treatment with the apparatus.

2. Background of the Related Art

Hair styling tools and devices are well known in the art and include heated combs, brushes, curlers, clippers, dryers, rollers and irons. Examples of these devices are disclosed in commonly assigned U.S. Pat. Nos. 7,490,615, 7,178,532, 7,082,949, 6,742,262, 6,671,460, 6,536,116, 6,191,387 and 4,731,519, U.S. Design Pat. No. D462,141, and U.S. Patent Publication Nos.: 2010/0180908 and 2005/0087205, the entire contents of each of these disclosures being hereby incorporated by reference herein. These devices or tools have proven to be quite effective for their intended uses.

### SUMMARY

The present disclosure is directed to further improvements in hair styling or treating tools. In accordance with one embodiment of the present disclosure, a hair treatment accessory for use with a hair styling apparatus includes a substrate base for mounting to a hair styling apparatus and a treatment agent disposed on the substrate base. The treatment agent is applied to one of the hair or skin of a subject during use of the hair styling apparatus. The treatment agent may include a conditioning, strengthening, repairing, hair shining or revitalizing fluid. The treatment agent may include conditioning oil. The conditioning oil may include ARGAN oil with or without silicon.

The substrate base may include an irregular surface dimensioned to assist in retention of the treatment agent on the substrate base. The irregular surface may be a porous structure including, e.g., open cells or channels. The irregular surface may be formed through a sintering process.

In accordance with another embodiment, a hair treatment apparatus includes a hair styling member for styling hair and an accessory member mounted to the hair styling member. The accessory member includes a substrate base and a treatment agent disposed on the substrate base. The treatment agent may include one of a conditioning, strengthening, repairing or revitalizing fluid material for application to hair of the subject. The substrate base may include a treatment surface having porosity for accommodating the treatment agent. The treatment agent may include Argan and silicone oil. The accessory member may be releasably mountable to the hair styling member. In one embodiment, the hair styling member may include a channel with the substrate base being dimensioned to be at least partially received within the channel with the treatment surface exposed for contacting the hair of the subject. The hair styling member may be one of a hair straighten, hair dryer, hair dryer attachment, hair curler, hair roller or hair brush.

The hair treatment apparatus may be incorporated within a package to provide a kit containing the hair styling member, and at least one of the accessory members. The kit may include a plurality of accessory members.

### BRIEF DESCRIPTION OF THE DRAWINGS

Various embodiments of the present disclosure are described hereinbelow with references to the drawings, wherein:

FIG. 1 is a perspective view of a hair treatment accessory in accordance with the principles of the present disclosure for incorporation with a hair styling apparatus;

FIG. 2 is an enlarged isolated view of the area of detail identified in FIG. 1 illustrating the substrate base surface of the hair treatment accessory;

FIG. 3 is a cross-sectional view of the hair treatment accessory taken along the lines 3-3 of FIG. 1;

FIG. 4 is a perspective view of a flat brush apparatus incorporating the hair treatment accessory of FIG. 1 in accordance with an embodiment of the present disclosure;

FIG. 5 is an isolated cross-sectional view illustrating one mechanism for mounting the hair treatment accessory to the flat brush apparatus of FIG. 4;

FIG. 6 is a perspective view of a cylindrical brush apparatus incorporating the hair treatment accessory of FIG. 1 in accordance with an embodiment of the present disclosure;

FIG. 7 is a perspective view of a hair straightener apparatus incorporating the hair treatment accessory of FIG. 1 in accordance with an embodiment of the present disclosure;

FIG. 8 is an isolated cross-sectional view illustrating one mechanism for mounting the hair treatment accessory to the hair straightener apparatus of FIG. 7;

FIG. 9 is a perspective view of another hair straightener apparatus incorporating the hair treatment accessory of FIG. 1 in accordance with an embodiment of the present disclosure;

FIG. 10 is a perspective view of a hair curling apparatus incorporating the hair treatment accessory of FIG. 1 in accordance with an embodiment of the present disclosure;

FIG. 11 is a cross-sectional view taken along the lines 11-11 of FIG. 10 illustrating one mechanism for mounting the hair treatment accessory to the curling apparatus of FIG. 10;

FIG. 12 is a perspective view of a hair dryer apparatus;

FIG. 13 is a perspective view of a comb attachment for the hair dryer apparatus of FIG. 12 incorporating the hair treatment accessory of FIG. 1 in accordance with an embodiment of the present disclosure;

FIG. 14 is a perspective view of a hair diffuser attachment for the hair dryer apparatus of FIG. 12 incorporating a hair treatment accessory in accordance with an embodiment of the present disclosure;

FIG. 15 is a perspective view of the hair treatment accessory for use with the hair diffuser attachment of FIG. 14;

FIG. 16 is a perspective view of a hair roller incorporating the hair treatment accessory in accordance with an embodiment of the present disclosure;

FIG. 17 is an enlarged cross-sectional view taken along the lines 16-16 of FIG. 17 illustrating one mechanism for mounting the hair treatment accessory to the hair roller of FIG. 16;

FIG. 18 is a view illustrating a kit incorporating a plurality of hair treatment accessories in accordance with an embodiment of the present disclosure.

### DETAILED DESCRIPTION OF THE EMBODIMENTS

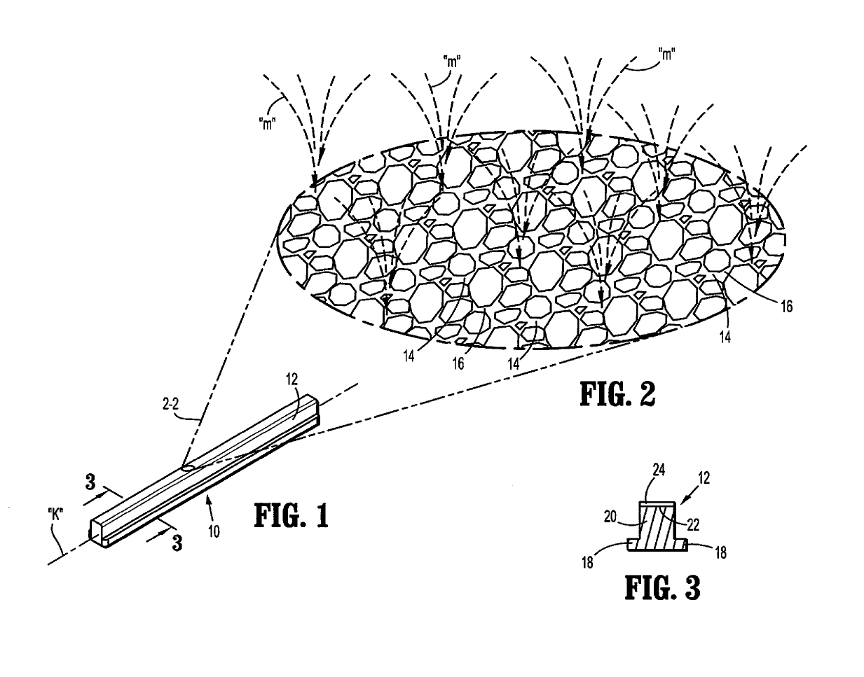

Referring to the drawings and, in particular, FIG. 1, there is shown a hair treatment accessory in accordance with the present disclosure. The hair treatment accessory 10 is to be incorporated within a hair treatment or styling apparatus, and may be integrally incorporated with the apparatus, or be an independent element secured to the apparatus after manufacture via various mounting arrangements to be discussed.

Hair treatment accessory 10 includes base substrate 12. Base substrate 12 may have various configurations depending on its intended application. In the embodiment of FIG. 1, base substrate 12 is elongated defining a longitudinal axis “k”; however, base substrate 12 is not limited to these shapes, but rather may circular, square, conical or any other geometric configurations to accommodate the hair treatment apparatus upon which the base substrate 12 will be secured or incorporated therewithin. Base substrate 12 may be made of various materials. In one embodiment best depicted in the detailed view of FIG. 2, base substrate 12 includes a porous material defining open pores or cells 14 and possibly channels 16 between the pores 14 for containing a hair treatment agent to be discussed. Such porous materials may include open celled plastics, metals and/or ceramics. In embodiments, the porous material may include sintered metals, plastics or the like. Examples of suitable materials include various sintered plastics such as sintered polypropylene, polyethylene, polytetrafluoroethylene (PTFE), polyvinylidene fluoride (PVDF) nylon or the like. The sintering process provides a porous hydrophilic surface and/or texture having cells or cavities which may be interconnected and exhibit defined wicking characteristics to permit the treatment agent to be retained within base substrate and then dispensed upon contact with the hair or skin of the subject. Examples of suitable sintered plastics are manufactured by POREX of Fairburn, Ga. The sintering process may be used to provide any number of shapes to base substrate 12.

In addition or as an alternative, base substrate 12 may comprise synthetic binding fibers molded into the desired configuration and adapted to produce wicking media with open-cell pore structures that control liquid volume capacity and fluid transfer rates. POREX produces various products incorporating this technology.

With reference now to FIGS. 1-3, base substrate 12 may be generally T-shaped in cross-section to facilitate mounting to the hair treatment apparatus. In accordance with this design, base substrate 12 defines lower winged segment with opposed wings 18 and upper linear segment 20. Upper linear segment 20 has upper treatment surface 22 which contacts the skin or hair of the subject during use of the hair styling apparatus. Upper treatment surface 22 may contain cells 14 and channels 16, i.e., having porosity or the open cell arrangement discussed hereinabove for retaining treatment agent 24. In FIG. 3, treatment agent 24 is represented as applied to treatment surface 22 although it is conceivable and desirable that treatment agent 24 may be contained within the open cells, pores and channels 14, 16 of treatment surface 24. In FIG. 2, treatment agent 24 is designated by arrows “m” directed towards cells 14 and channel 16. Treatment surface 24 may alternatively include an irregular surface created through knurling, chemical deposition processes or other mechanical means. An irregular surface may provide more surface area and surface tension for assist in retaining treatment agent 24 on the treatment surface 22. Retentive or hydrophilic coatings may also be applied to treatment surface 22 to assist in retaining treatment agent 22 or treatment surface 22.

In one embodiment, treatment agent 24 includes argan oil. Argan oil is extracted from the fruits of the argan tree, argania spinosa, that is endemic to Morocco. The hair care composition may solely contain argan oil, or may include argan oil in combination with other ingredients. Examples of other ingredients include pharmaceutically active agents, moisturizers, hydration agents, penetration agents, preservatives, emulsifiers, natural or synthetic oils, solvents, surfactants, detergents, gelling agents, emollients, antioxidants, fragrances, fillers, thickeners, waxes, odor absorbers, dyestuffs, coloring agents, powders, viscosity-controlling agents, buffers, protectants, pH regulators, chelating agents, humectants, conditioners, glitter, mica, minerals, silicones, polyphenols, sunblocks, phytomedicinals, and combinations thereof, as well as other additives typically used in hair care products as appreciated by those skilled in the art.

In embodiments, the hair care composition may include argan oil and emollients and/or conditioning agents, alone or in combination with other ingredients as discussed above. In embodiments, the hair care composition includes argan oil and silicone. Silicone includes, for example, silicone oils and oils having a hydrocarbon backbone, silicone oils combining cyclic polydimethylsiloxanes, α,ω-hydroxylated polydimethylsiloxanes, α,ω-trimethylsilyl polydimethylsiloxanes, polyorganosiloxanes such as polyalkylmethylsiloxanes, polymethylphenylsiloxanes, polydiphenylsiloxanes, aminosilicone derivatives, silicone waxes, copolyether silicones (such as the oil MIRASIL DMCO sold by Rhone-Poulenc, or DC 190 sold by Dow Coming) or mixed silicone derivatives including various types of derivatization (such as polyalkylmethyl-siloxane/copolyether silicone mixed copolymers). An argan/silicon conditioning agent may strengthen, repair or condition hair, while potentially adding shine to the hair.

Other suitable emollients include, for example alkylmonoglycerides, alkyldiglycerides, and/or triglycerides such as oils extracted from plants and vegetables (palm oil, coconut oil, cotton seed oil, soybean oil, sunflower oil, olive oil, grape seed oil, sesame oil, ground nut oil, castor oil, combinations thereof, and the like), oils of marine origin (fish oils, etc.) and derivatives of these oils, such as hydrogenated oils, lanolin derivatives, mineral oils or paraffinic oils, perhydrosqualane, squalene, diols such as 1,2-propanediol and 1,3-butanediol, cetyl alcohol, stearyl alcohol, oleyl alcohol, polyethylene glycols or polypropylene glycols, and fatty esters such as isopropyl palmitate, 2-ethylhexyl cocoate, myristyl myristate, esters of lactic acid, stearic acid, behenic acid, isostearic acid.

In embodiments, the hair care composition may include argan oil and conditioners, alone or in combination with other ingredients. Conditioners include, for example, those of natural or synthetic origin, such as those known under the generic CTFA name “Polyquaternium”, for instance the MIRAPOL A15® or MIRAPOL 550® polymers from Rhone-Poulenc, cationic polysaccharide derivatives (cationic derivatives of cellulose, of guar or of carob), such as cocodimonium hydroxyethyl cellulose, guar hydroxypropyl trimonium chloride, hydroxypropyl guar hydroxypropyl trimonium chloride (JAGUAR C13S®, JAGUAR C162® sold by Rhone-Poulenc), volatile or non-volatile silicone derivatives, for instance amodimethicone, cyclomethicones, water-insoluble, non-volatile polyorganosiloxanes, for instance oils, resins or gums, such as diphenyldimethicone gums, combinations thereof, and the like.

Examples of other additives which may be useful in the hair care composition include additives for promoting moisturization of the hair and/or skin (wetting agents), for instance certain carbohydrates (for example glycerol or sorbitol), polyethylene glycols or polypropylene glycols, alkoxylated derivatives of sugars or of sugar derivatives (for example methylglucose), water-soluble or water-dispersible polymers such as collagen or certain non-allergenic derivatives of marine or plant proteins (for example wheat protein hydrolysates). Thickeners, such as natural hydrocolloids (guar gum, carob gum, tara gum, etc.) or hydrocolloids derived from fermentation processes, such as xanthan gum, polysaccharides extracted from seaweed, such as carrageenans, and polycarbohydrate derivatives such as modified celluloses (for example hydroxyethylcellulose, carboxymethylcellulose), or nonionic derivatives (for example hydroxypropylguar), anionic derivatives (carboxymethylguar) or nonionic/anionic mixed derivatives, such as carboxy-hydroxypropyl-guars or nonionic/cationic derivatives, can also be present.

The hair care composition may be in the form of a solution, emulsion, suspension, cream, lotion, gel, powder, foam, or other solid or liquid composition suitable for topical application to hair and skin.

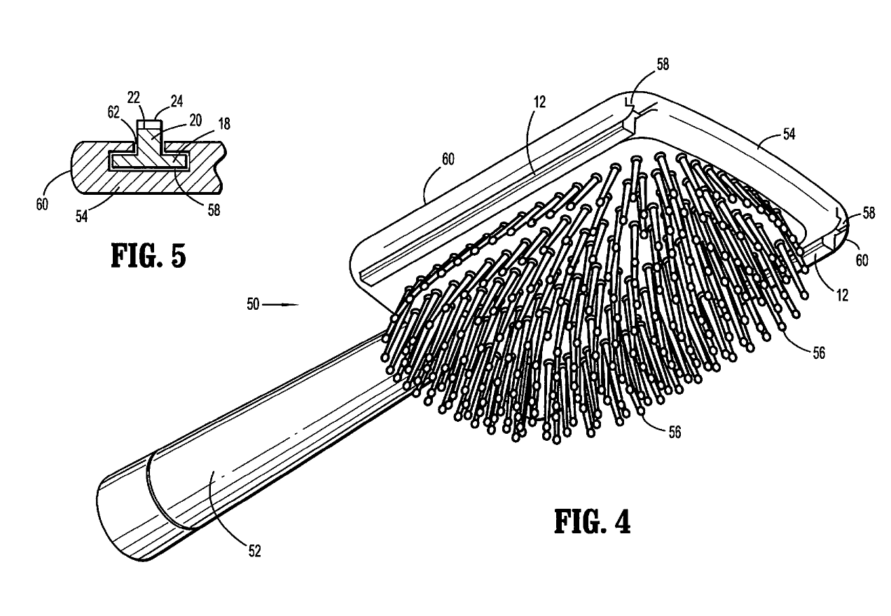

FIG. 4 illustrates a hair treatment apparatus in the form of a flat brush apparatus 50 which incorporates hair treatment accessory 10. Flat brush apparatus 50 includes brush handle 52 and brush base 54 connected to the handle 52. Brush base 54 has a plurality of bristle elements 56 depending therefrom for passage through the hair of the subject. Base 54 includes longitudinal channels 58 adjacent opposed edges 60. As best depicted in the view of FIG. 5 which is a cross-section of base 54 encompassing channels 58, the channels 58 are generally “t-shaped” in configuration to correspond to the general “t-shaped” cross-section of base substrate 12 of hair treatment accessory 10. For example, wings 18 of each base substrate 14 may be slid within the open end of channels 58 to assume the position depicted in FIGS. 4 and 5, thereby mounting treatment accessory 12 to brush base 54. For example, wings 18 may be confined within channels 58 with linear segment 20 extending through longitudinal slot 62. Other alternate means for mounting treatment accessory 10 to brush base 54 are envisioned including with the use of adhesives, loop and hook arrangements (VELCRO), snap fit, bayonet couplings or the like. In the alternative, hair treatment accessory 10 may be integrally and/or monolithically formed with brush base 54. In use of flat brush apparatus 50 in combing or brushing hair of the patient, as brush base 54 and bristles elements 56 pass through the hair, at least upper surface 22 of base substrate 12 contacts the hair, which, in turn causes release of treatment agent 24 onto the hair to achieve the desired therapeutic or cosmetic affect.

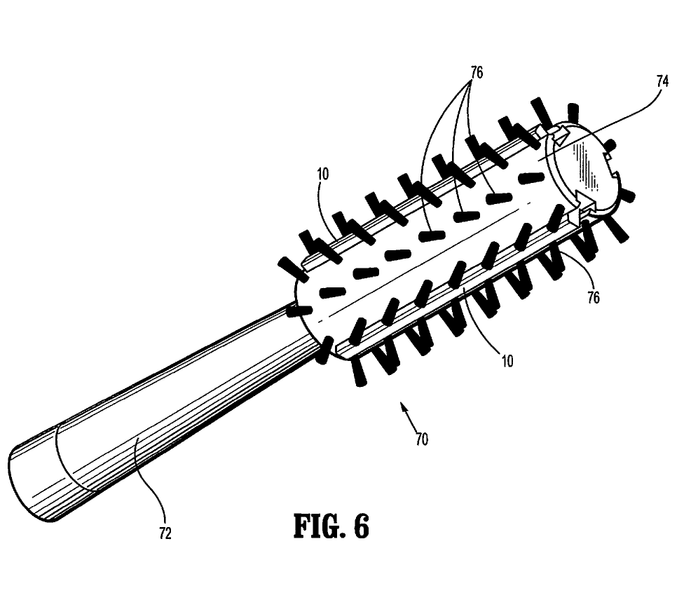

FIG. 6 illustrates another hair treatment apparatus in the form of a cylindrical brush apparatus, which incorporates hair treatment accessory 12. Cylindrical brush apparatus 70 includes brush handle 72 and cylindrical brush 74 connected to the brush handle 72. Brush 74 has a plurality of bristles elements 76 depending from the circumference thereof, which, may or may not be arranged in axial rows of bristle elements 76. Between at least two adjacent rows of bristle elements 76 is hair treatment accessory 10, which may be mounted to cylindrical brush 74 through any of the aforementioned mounting means e.g., through the channel arrangement depicted in FIGS. 4-5. In embodiments, at least two or more hair treatment accessories 10 are provided between adjacent rows of bristle elements 16. In one embodiment, hair treatment accessories 10 are radially spaced about the entirety of the perimeter or circumference of brush head 74 to ensure that the hair or skin of the subject is treated with treatment agent 24 regardless of the radial orientation of the brush head 74.

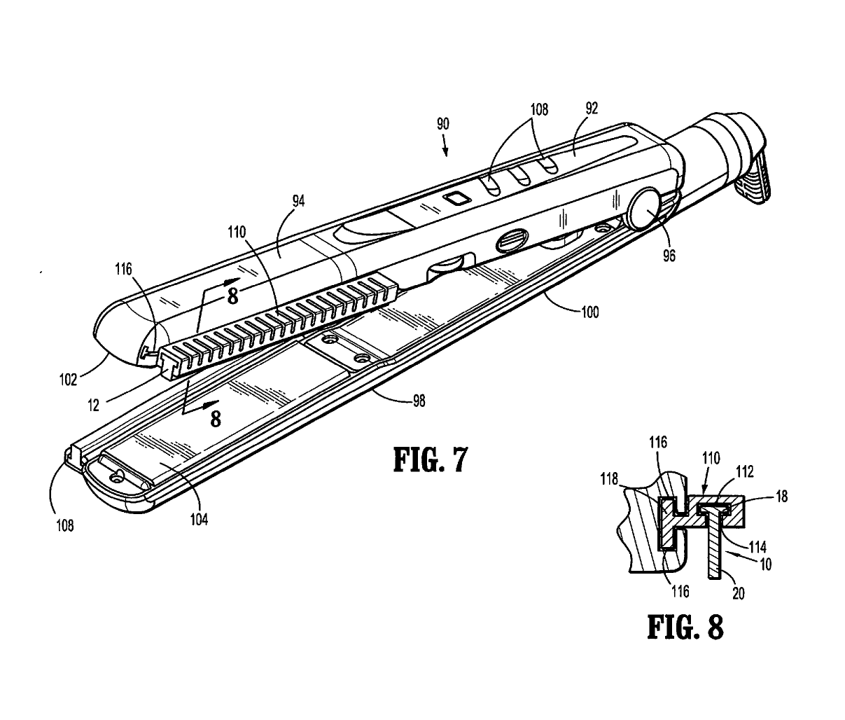

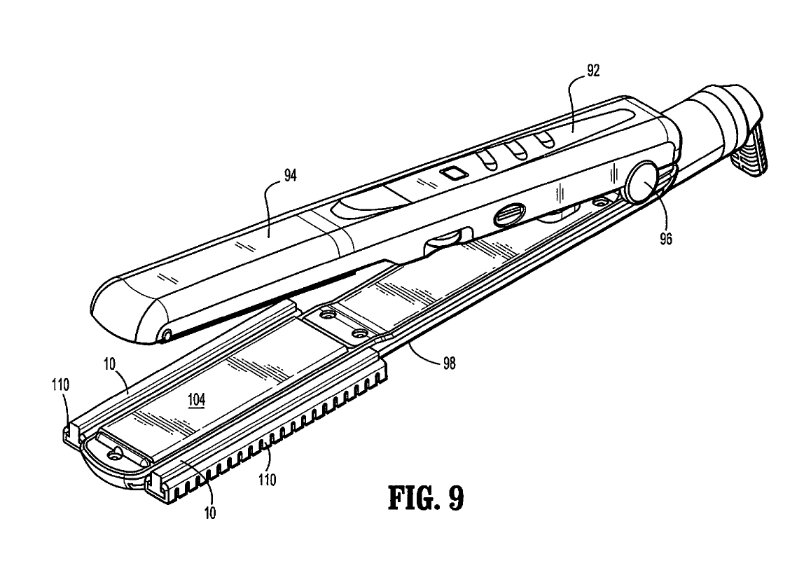

FIG. 7 illustrates a hair treatment apparatus in the form of a hair straightener or iron apparatus 90 which incorporates hair treatment accessory 10. Hair straightener apparatus 90 may be of the type disclosed in commonly assigned U.S. Pat. No. 7,178,532 previously incorporated by reference herein. Apparatus 90 includes handle portion 92 and head portion 94 joined by spring-biased hinge 96, of a conventional type, to another head portion 98 and handle portion 100. Handle portions 92, 100 are biased away from each other by the spring-biased hinge 96, as is known in the art. Each head portion 94, 98 has heatable plate 102, 104 heated by conventional electrical means (not shown) known in the art, so that hair can be positioned therebetween for styling. Handles 92, 100 can contain control buttons 108 to operate the device 10. As best depicted in FIGS. 7 and 8, each head portion 94, 98 may include an extension 110 separately attached or integrally formed with the respective head portion 94, 98. Extension 110 is dimensioned to accommodate base substrate 12 of hair treatment accessory 10 in a manner to present treatment surface 22 of the base substrate 12 to hair passing through the head portions 94, 98. In this embodiment, linear segment 20 may have a height which is greater than the height of the corresponding linear segment 22 of the embodiment of FIG. 1. In one embodiment, extension 110 defines a rectangular channel 112 for reception of wings 18 of base substrate 12 with linear segment 20 extending through slot 114 of extension 110. In one embodiment, each head portion 94, 98 includes a generally t-shaped groove 116 in a side of the respective head portion 94, 98. Groove 116 accommodates a correspondingly dimensioned “t-shaped” mount 118 depending from extension 110 to permit the extension 110 to be slid within the groove 114 to thereby releasably mount the extension 110 and hair treatment accessory 10 to apparatus 90. Extension 110 may be connected to head portions 94, 98 through any other conventional means including adhesives, tongue/groove arrangements, VELCRO, or may be integrally formed with the head portions. In the embodiment of FIGS. 7 and 8, extensions 110 and heat treatment accessories 10 are on opposed sides or edges of respective head portions 94, 98. FIG. 9 illustrates an alternate embodiment where extensions 110 and head treatment accessories 10 are mounted on opposed sides of a single head portion 94. Any of the aforementioned mounting means for effecting the mounting may be utilized. It is also envisioned that extensions 110 and accessories 10 may be mounted on each side of each head portion 94, 98 such that four accessories 10 are provided.

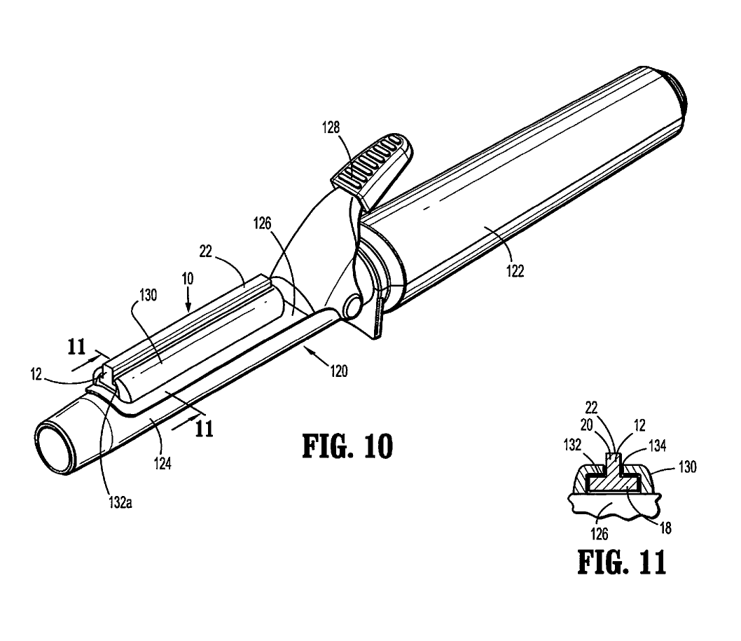

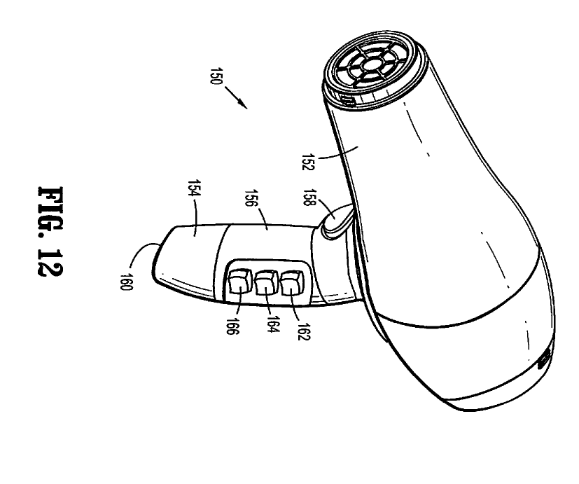

FIGS. 10-11 illustrate a hair treatment apparatus in the form of a hair curling iron apparatus 120 which incorporates hair treatment accessory 10. Hair curling iron apparatus 120 may be of the type disclosed in commonly assigned U.S. Pat. No. 4,731,519 previously incorporated herein by reference. Curling iron apparatus 120 includes handle 122 and cylindrical barrel 124. Handle 122 is of a generally cylindrical shape and barrel 124 is in axial alignment with it. Hair clamp or spoon 126 is spring-pressed against a portion of barrel 124 and is controlled by spring-pressed control lever 128. Electrical resistance elements are provided for heating barrel 124. Hair clamp 126 may include extension 130 extending along the length of the exterior of the clamp. Extension 130 may be mounted to the exterior of clamp 126 by conventional means or integrally formed with the clamp 126. Extension 130 may be releasably attached to clamp 126 via any of the aforementioned means or may be integrally formed with clamp 126. Extension 130 includes longitudinal channel 132 of rectangular cross-section and longitudinal slot 134 in communication with the longitudinal channel 132. Longitudinal channel 132 receives lower winged segment 18 of base substrate 12 with upper linear segment 20 extending through longitudinal slot 134. Base substrate 12 may be positioned within extension 130 via sliding lower winged segment 18 through the open end of longitudinal channel 132 of the extension 130. Treatment agent 24 contained within or on upper treatment surface 22 is dispensed on the hair or skin of the patient during application of the curling apparatus 120 in treating/curling hair.

FIG. 12 illustrates a hair treatment apparatus in the form of a hair dryer apparatus 150 which incorporates hair treatment accessory 10. Hair dryer apparatus 150 may be of the type disclosed in commonly assigned U.S. Pat. No. 6,671,460 previously incorporated herein by reference. Hairdryer apparatus 150 includes body 152, handle or hand grip 154, barrel 156 common to several controls, a master switch 158 and connection 160 for the power lead (not depicted). An array of controls having three switches 162, 164, 166 has been depicted. In general, a first switch 162 allows the speed of the air flow leaving the blower to be adjusted, in this instance by varying the rotational speed of this blower. A second switch 164 allows the heating of the air to be adjusted. A third switch 166 may be used, for example, as a master switch or may fulfill some other additional function. Other arrangements are also envisioned.

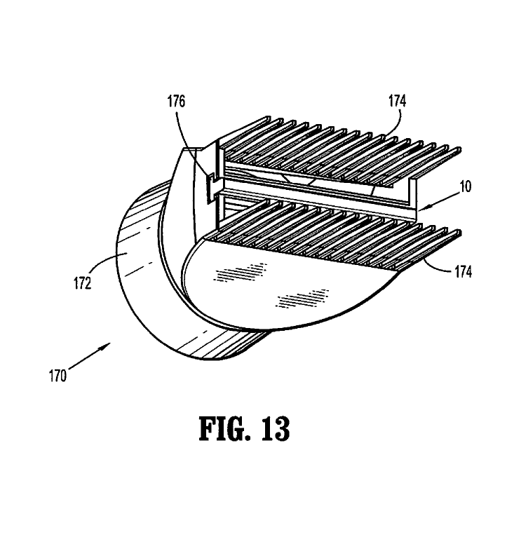

FIG. 13 illustrates a comb attachment 170 for attaching to body 152 of hair dryer apparatus 150. Comb attachment 170 includes cylindrical base 172 for positioning over the end of body 152 of hair dryer apparatus 150, and a pair of parallel combs 174 depending from the base 172. Comb base 172 may be releasably mountable to dryer body 152 through any conventional means including a friction or tolerance fit, snap-fit, bayonet coupling or the like. Disposed between combs 174 is hair treatment accessory 10 which is positioned releasably or permanently fixed within a correspondingly dimension channel 176 of comb base 172. Channel 176 may be similar to the arrangement discussed in the prior embodiments. In this position, hair treatment accessory 10 will dispense hair treatment agent 24 as the hair is passed through the parallel combs 174.

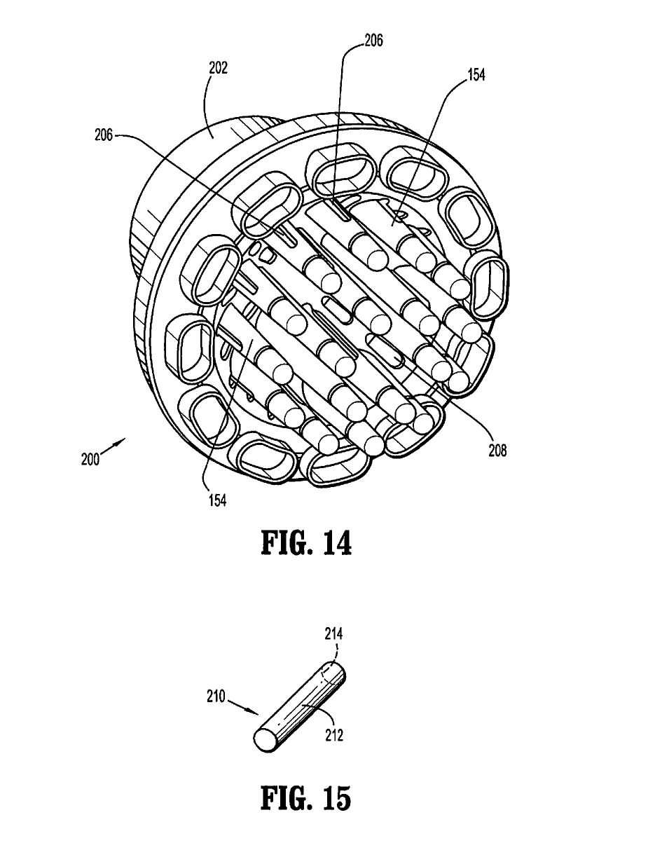

FIG. 14 illustrates an alternate attachment for the hair dryer apparatus 150 of FIG. 12 and an alternate arrangement of hair treatment accessory 10. Dryer attachment 200 is in the form of a hair diffuser adapted to provide a volumizing effect on the hair. Hair diffuser 200 includes diffuser base 202 which is attachable to body 152 of hair dryer apparatus 150 and a plurality of fingers 204 depending from the diffuser base 202. Diffuser base 202 may be mounted to body 152 in a manner similar to base 172 of comb attachment 170 of FIG. 13. Fingers 204 may define open channels in fluid communication with the interior of diffuser base 202 to direct heated air emitted by the hair dryer apparatus 150 through the fingers 204. Alternatively, fingers 204 may be closed whereby hair is emitted through side openings 206 in the fingers 204or openings 208 in diffuser base 202. With reference to FIG. 15, in this embodiment, hair treatment accessory 210 is dimensioned for mounting to fingers 204 and may define a cylindrical or conical sleeve base substrate 212 which is positioned over the ends of fingers 204. Base substrate 212 may be fabricated in accordance with the methodology described in connection with hair treatment accessory 10 of FIG. 1. Hair treatment agent is dispensed on or within the outer surface of base substrate 212. In one embodiment, base substrate 202 establishes a tolerance fit with each finger 204 of diffuser base 202 to mount the hair treatment accessory 10 to the respective finger 204. The extreme ends of base substrate 212 may be open for use with open ended fingers 152 or closed for use, e.g., with closed end fingers 152. In FIG. 15, the extreme end of base substrate 212 is closed but may be open as illustrated by phantom lines 214. During use, hair passing through fingers 204 and accessory 210 is subjected to treatment agent 24 for distribution on hair as discussed hereinabove,

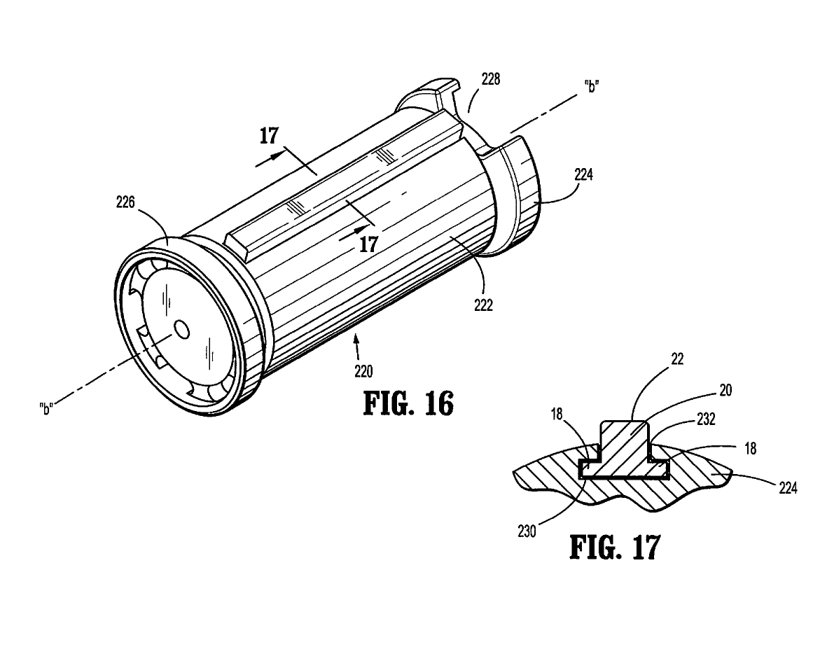

FIG. 16 illustrates a hair roller which may incorporate hair treatment accessory of the present disclosure. Hair roller 220 includes roller base 222 defining longitudinal axis “b” and opposed roller flanges 224, 226 on each side of the roller base 222. Flange 224 has a recessed or gapped section 228 which permits access to internal channel or groove 230. Groove 230 accommodates hair treatment accessory 10. Hair treatment accessory 10 is similar to the hair treatment accessory 10 described in connection with FIGS. 1-3. In one embodiment depicted in FIG. 17, wings 18 slide through recessed section 228 of flange 224 to be received within correspondingly dimensioned channel 230 with linear segment 20 extending through external longitudinal slot 232 which may extend the length of groove 230. In use, hair is wrapped about roller base 222 whereby treatment agent 24 on exposed treatment surface 22 applies the conditioning, revitalizing, repair, strengthening and/or shining effect to the hair.

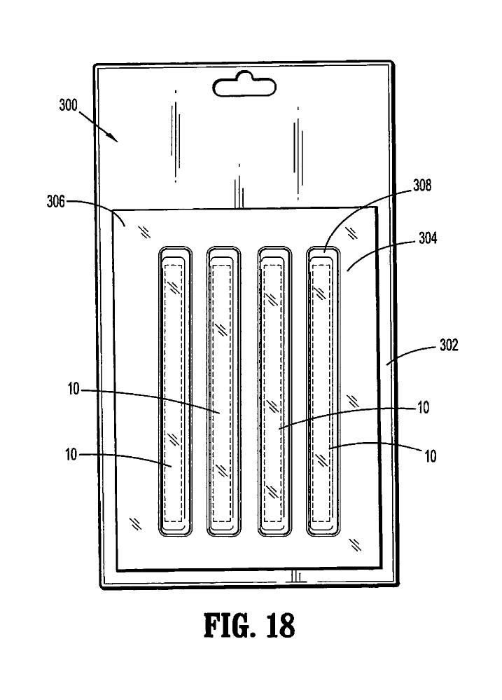

FIG. 18 illustrates a kit 300 incorporating a plurality of hair treatment accessories 10 contained within a package 302. Each hair treatment accessory 10 may be disposed after a single or multiple use and discarded. Thereafter, a new hair treatment accessory 10 may be mounted to the desired hair treatment apparatus for a subsequent use. Package 302 may be any suitable package adapted to contain a predetermined number of hair treatment accessories, including foil packs, blister packs, cardboard arrangements or the like. In one embodiment, package 302 includes a foil 304 mounted to package base 306. Each treatment accessory 10 may be removed individually from package 302 by removing a portion of the coil covering the package. In this manner, foil 304 remains in tact covering the remaining treatment accessories 10 and providing an air tight enclosure containing each accessory to preserve the treatment agent 24. Package 302 may have predefined channels 308 for individually accommodating respective treatment accessories 10. It is also envisioned that each hair treatment accessory 10 may be stored within a separate air tight package such as a foil wrap to assist in retaining the hair treatment agent 24 within the base substrate 12 and minimize the potential of the agent 24 drying or releasing from the substrate. Package 302 may also include treatment accessories 210 of FIG. 15 with or without treatment accessories 10.

Although the illustrative embodiments of the present disclosure have been described herein with reference to the accompanying drawings, the above description, disclosure, and figures should not be construed as limiting, but merely as exemplifications of particular embodiments. It is to be understood, therefore, that the disclosure is not limited to those precise embodiments, and that various other changes and modifications may be effected therein by one skilled in the art without departing from the scope or spirit of the disclosure.

1. A hair treatment accessory for use with a hair styling apparatus having hair contact elements for contacting and styling hair, which comprises:
a substrate base for mounting to a hair styling apparatus, the substrate base including structure dimensioned and adapted to mount the substrate base to the hair styling apparatus independent of the hair contact elements thereof; and
a treatment agent disposed on the substrate base, the treatment agent being applied to one of hair or skin of a subject during use of the hair styling apparatus

2. The hair treatment accessory in accordance with claim 1 wherein the treatment agent includes a conditioning, strengthening, repairing or revitalizing fluid.

3. The hair treatment accessory in accordance with claim 2 wherein the treatment agent includes a conditioning oil.

4. The hair treatment accessory in accordance with claim 3 wherein the conditioning oil includes ARGAN oil.

5. The hair treatment accessory in accordance with claim 4 wherein the conditioning oil includes silicon.

6. The hair treatment accessory in accordance with claim 2 wherein the substrate base includes an irregular surface dimensioned to assist in retention of the treatment agent on the substrate base.

7. The hair treatment accessory in accordance with claim 6 wherein the irregular surface includes open cells or channels.

8. The hair treatment accessory in accordance with claim 7 wherein the irregular surface is formed through a sintering process.

9. A hair treatment apparatus which comprises:
a hair styling member for styling hair, the hair styling member including one of bristles or teeth dimensioned and arranged for contacting and styling the hair; and
an accessory member mounted to the hair styling member independent of the one of the bristles or teeth, the accessory member including a substrate base and a treatment agent disposed on the substrate base, the treatment agent including one of a conditioning, strengthening, repairing or revitalizing fluid material for application to hair of the subject.

10. The hair treatment apparatus in accordance with claim 9 wherein the substrate base includes a treatment surface, the treatment surface having porosity for accommodating the treatment agent.

11. The hair treatment apparatus in accordance with claim 10 wherein the treatment agent includes Argan and silicone oil.

12. The hair treatment apparatus in accordance with claim 10 wherein the accessory member is releasably mountable to the hair styling member.

13. The hair treatment apparatus in accordance with claim 12 wherein the hair styling member includes a channel and wherein the substrate base is dimensioned to be at least partially received within the channel with the treatment surface exposed for contacting the hair of the subject.

14. The hair treatment apparatus in accordance with claim 9 wherein the hair styling member is one of a hair straighten, hair dryer, hair dryer attachment, hair curler, hair roller, hair brush.

15. The hair treatment apparatus in accordance with claim 14 including a package to provide a kit containing the hair styling member and at least one of the accessory member.

16. The hair treatment apparatus in accordance with claim 15 wherein the kit includes a plurality of accessory members.

17. The hair treatment accessory in accordance with claim 1 wherein the substrate base is substantially rigid comprising a ceramic, metallic or rigid polymeric material.

18. The hair treatment accessory in accordance with claim 17 wherein the substrate base comprises a ceramic or metallic material.

19. The hair treatment accessory in accordance with claim 18 wherein the substrate base includes wherein the substrate base includes an irregular surface outer surface dimensioned to assist in retention of the treatment agent on the substrate base.

20. A method for treating hair, comprising the steps of:
providing a hair styling member having one of bristles or teeth dimensioned and arranged for contacting and styling hair;
mounting an accessory member to the hair styling member independent of the one of the bristles or teeth, the accessory member including a substrate base and a treatment agent disposed on the substrate base, the treatment agent including one of a conditioning, strengthening, repairing or revitalizing fluid material for application to hair of the subject; and
combing or brushing the hair with the hair styling member such that the one of the bristles or teeth passes through the hair while the treatment agent of the accessory member is applied to the hair for treatment.
